(19) United States
(12) Patent Application Publication
(10) Pub. No.: US 2026/0085268 A1
(43) Pub. Date: Mar. 26, 2026

(54) APPARATUS AND METHODS FOR ELECTROPORATION

(71) Applicant: MaxCyte, Inc., Rockville, MD (US)

(72) Inventors: Sergey Mikhailovych DZEKUNOV, Germantown, MD (US); Nicholas CHOPAS, Germantown, MD (US); Stephen PORTER, Baltimore, MD (US)

(73) Assignee: MaxCyte, Inc., Rockville, MD (US)

(21) Appl. No.: 19/109,342

(22) PCT Filed: Sep. 6, 2023
(86) PCT No.: PCT/US2023/073545

§ 371 Date: Mar. 6, 2025

Related U.S. Application Data
(60) Provisional application No. 63/404,157, filed on Sep. 6, 2022.

(51) Int. Cl. C12M 1/42; C12M 1/36; C12N 13/00

(52) U.S. Cl. CPC C12M 35/02; C12M 41/48; C12N 13/00

(57) ABSTRACT

An apparatus and methods for electroporation of cells are disclosed. The apparatus comprises a memory storing instructions, a high voltage module to apply a voltage to cells, a low voltage module including a user interface, a system board connected to the high voltage module, the low voltage module, and a single board computer, and the single board computer including at least one processor to control the high voltage module and the low voltage module. The system board includes an emergency stop button and an electronic circuit. The electronic circuit receives a signal from the emergency stop button, latches the signal from the emergency stop button, blocks a logical signal controlling the voltage delivered to the instrument output, terminates the voltage applied to cells, and disables the at least one processor to provide commands to the high voltage module.

### PRIORITY

This application claims priority to U.S. Provisional Application No. 63/404,157 filed on Sep. 6, 2022, which is incorporated herein by reference in its entirety.

### TECHNICAL FIELD

The present disclosure generally relates to apparatus, construction, and methods for electroporation. In particular, embodiments of the present disclosure relate to inventive and unconventional apparatus and methods providing the electroporation of cells in a cell suspension from a fluid sample where the apparatus integrates a display and touch screen, a computer, application of high voltage to cells in the cell suspension from the fluid sample, and a non-software based emergency stop feature.

### BACKGROUND

Electroporation systems may apply a voltage to cells in a cell suspension in a fluid through an electric field in order to increase the permeability of the cells'membrane allowing chemicals, drugs, or nucleic acids (DNA or RNA) to be introduced in the cells. The application of a voltage may result in the electric field following a pulsing duration schedule or protocol. Electroporation systems may be used in medical, testing, and microbiology applications.

Current electroporation systems may be standalone systems that may only be designed for transfection, which may necessitate the purchase of separate desktop or laptop computers to process results from the current electroporation systems. For example, operators or users of current electroporation systems may be required to additionally purchase desktop or laptop computers to analyze and generate plots from current electroporation systems where the current electroporation systems may only provide operators or users with raw data. The non-integrated electroporation system necessitating the purchase of separate desktop or laptop computers may, additionally, create problems with the compatibility between the desktop or laptop computers'operating systems and the electroporation system. For example, current electroporation systems may provide operators or users with a separate software to install on desktop or laptop computers in order to process the raw data generated by the current electroporation systems where the software may help generate plots, identify, and analyze transfected cells in a cell suspension in a fluid. In other instances, the software provided for installing on desktop or laptop computers may not be compatible or up to date with existing operating systems, which may force operators or users to maintain older versions of desktop or laptop computers'operating system. Moreover, current electroporation system may require the use of separate storing mediums for receiving results of the transfection process in the form of a file containing raw data, which may in turn be used on a desktop or laptop computer for processing the file for results. The use of a separate storing medium may also necessitate the use of a software to process the results in the file.

The non-integration of computer functionality in current electroporation systems significantly increases costs to operators or users because they may be forced to purchase standalone electroporation systems and desktop or laptop computers and to incur costs with converting current operating systems on desktop or laptop computers to old operating systems compatible with the electroporation systems'provided software. Furthermore, the non-integration of computer functionality may also increase the difficulty in operating those systems because operators or users may have to rely on the systems'operating manual to understand the functions of outmoded mechanical buttons or switches. For example, current electroporation systems may not utilize a display screen to guide operators or users along the transfection process or may not provide a status of the transfection process. For example, manufacturers of current electroporation systems may rely on standalone software for installation on desktop or laptop computers to provide operators or users with the electroporation status; however, those status may be delayed because of the non-integration between the electroporation system, the desktop or laptop computers, and software.

Furthermore, the non-integrated current electroporation systems with desktop or laptop computers may not provide a safety feature that allows an operator or user to immediately terminate a transfection process when the operator or user may discover hazardous conditions with a malfunctioning electroporation system, the wrong fluid sample ready for processing, a flammable fluid sample, or a spill of conductive fluid sample inside or outside of the electroporation system, which may create dangerous conditions for operators or users. For example, operators or users may be forced to unplug the electroporation system all together or disconnect the desktop or laptop computers from the electroporation system, which may also pose highly hazardous conditions because of the chance of electrocution or fires. A non-integrated electroporation system may allow for operators or users to use a command on a desktop or laptop computer, such as the keyboard's space-bar, to terminate the electroporation process; however, this implementation through the use of the non-integrated electroporation systems' desktop or laptop computers may not be instantaneous because the operator may have to drive through a series of menus before the software recognizes that the use of the keyboard's space-bar may mean to terminate the electroporation process. Moreover, the space-bar application for terminating the electroporation process may be software dependent where software may be limited by errors, delays, and steps of logical sequences that may increase the time to execute the termination of the electroporation system. In addition, the software-based space-bar application for terminating the electroporation process may be susceptible to error in communications between desktop or laptop computers and current electroporation systems.

The lack of a means for immediately terminating a transfection process increases cost to operators and users because of the lost electroporation system, the lost desktop or laptop computers, the lost fluid samples, or serious injuries to operators and users.

Therefore, there is a need for improved apparatus and methods for electroporation of fluids that integrates computer functionality, applies a higher strength of the electric field Volt per meter, applies to higher volumes of fluid ranging up to 1 litter (1 L), applies to higher culture size, and incorporates a non-software-based emergency stop feature to immediately terminate the electroporation process to preserve fluid samples, electroporation apparatus, and operator and users.

### SUMMARY

One aspect of the present disclosure is directed to an apparatus for electroporation of cells in a cell suspension in a fluid. The apparatus may include a memory storing instructions, a high voltage module including an instrument output configured to apply a voltage to cells in a cell suspension in a fluid, a low voltage module including a user interface device, a system board connected to the high voltage module, the low voltage module, and a single board computer, the single board computer including at least one processor configured to (i) send instructions to control the high voltage module and the low voltage module, and (ii) receive instructions from the user interface device to regulate the voltage applied by the instrument output to cells in the cell suspension in the fluid, an emergency stop button connected to the system board. The system board may further include an electronic circuit that may be configured to execute the instructions to perform steps comprising of receiving a signal from the emergency stop button to stop the voltage applied by the instrument output to the cells in the cell suspension in the fluid, latching the signal from the emergency stop button, blocking a logical signal that controls voltage delivery to the instrument output, terminating the voltage applied by the instrument output to the cells in the suspension of the fluid; and disabling the at least one processor from providing instructions to the high voltage module.

Another aspect of the present disclosure is directed to a method of performing a safety step terminating the electroporation of cells in a cell suspension in a fluid. The method may comprise the steps of receiving a signal from an emergency stop button to stop the voltage applied by an instrument output to cells in a cell suspension in a fluid, latching the signal from the emergency stop button, blocking a logical signal that controls voltage delivery to the instrument output, terminating the voltage applied by the instrument output to the cells in the cell suspension in the fluid, and disabling the at least one processor from providing commands to the high voltage module.

Yet another aspect of the present disclosure is directed to an apparatus for electroporation of cells in a cell suspension in a fluid. The apparatus may include a memory storing instructions, a high voltage module including an instrument output configured to apply a voltage to cells in a cell suspension in a fluid, a low voltage module including a user interface device, a system board connected to the high voltage module, the low voltage module, and a single board computer, the single board computer including at least one processor configured to (i) send instructions to control the high voltage module and the low voltage module, and (ii) receive instructions from the user interface device to regulate the voltage applied by the instrument output to the cell in the cell suspension in the fluid, an emergency stop button that may be connected to the system board, and a D flip-flop in the system board that may have a preset input pin connected to the emergency stop button, an output pin and a clock input pin connected to the low voltage module, and an inverted output pin connected to the high voltage module. The D flip-flop in the system board may be configured to execute the instructions to perform steps including receiving a signal from the emergency stop button to stop the voltage applied by the instrument output to the cells in the cell suspension in the fluid, latching the signal from the emergency stop button, blocking a logical signal that controls voltage delivery to the instrument output, terminating of the voltage applied by the instrument output to the cells in the cell suspension in the fluid, and disabling the at least one processor from providing commands to the high voltage module.

Other systems, apparatus, and methods are also discussed herein.

### BRIEF DESCRIPTION OF THE DRAWINGS

FIG. 1 is a schematic block diagram illustrating an exemplary embodiment of an integrated electroporation apparatus with non-software-based emergency stop, consistent with the disclosed embodiments.

FIG. 2 is a schematic block diagram illustrating an exemplary non-software-based emergency stop feature, consistent with the disclosed embodiments.

FIG. 3 depicts an illustration of a graphical user interface of a display and touch screen for resetting the emergency stop signal, consistent with the disclosed embodiments.

FIG. 4 is an exemplary illustration of the integrated electroporation system and its components in the static electroporation and flow electroporation configurations, consistent with the disclosed embodiments.

FIG. 5 is an exemplary illustration of a processing assembly interfacing an instrument output in the static electroporation configuration, consistent with the disclosed embodiments.

FIG. 6 is an exemplary illustration of a processing assembly interfacing an instrument output in the flow electroporation configuration, consistent with the disclosed embodiments.

FIG. 7 is an exemplary illustration of the electroporation system with the packaging of low voltage components and the display and touch screen, consistent with the disclosed embodiments.

FIG. 8 illustrates exemplary block diagram for an exemplary method for electroporation of cells in a cell suspension in a fluid, consistent with some embodiments of the present disclosure.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions, or modifications may be made to the components and steps illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope of the invention is defined by the appended claims.

Embodiments of the present disclosure are directed to apparatus and methods for electroporation configured to apply a maximum voltage to cells in a cell suspension in a fluid sample for increased transfection yield due to increase permeability of cells' membrane for the introduction of chemical or biological samples. Furthermore, embodiments of the present disclosure are directed to apparatus and methods for electroporation configured to transfect cells in cell suspension for volumes of fluids of, for example, 1 L containing a culture size of, for example, 200 billion cells. Moreover, embodiments of the present disclosure are directed to apparatus and methods for electroporation configured to integrate computer functionality including a display and touch screen without the need to utilize standalone desktop or laptop computers for ease of use. Furthermore, embodiments of the present disclosure are directed to apparatus and methods for electroporation configured to include an emergency or safety stop feature that is not software-based and provides immediate termination of the electroporation process to reduce cell destruction, apparatus destruction, and the safety of operators or users.

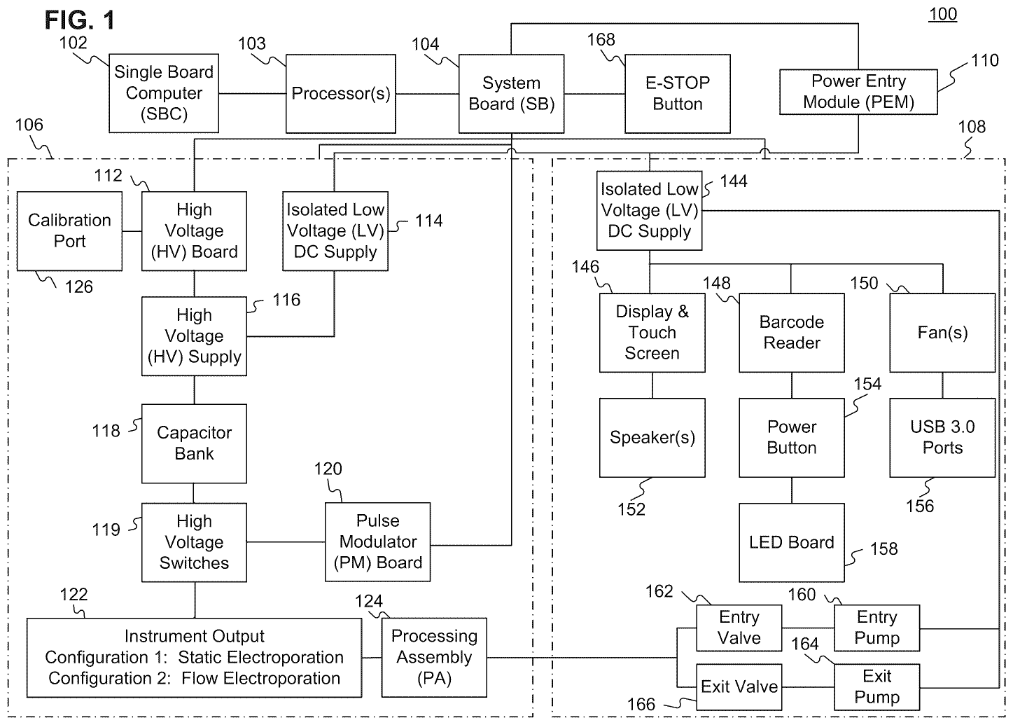

FIG. 1 is a schematic block diagram illustrating an exemplary embodiment of an integrated electroporation apparatus with a non-software-based emergency stop, consistent with the disclosed embodiments. As illustrated in FIG. 1, system 100 may include one or more devices—components—that may constitute system 100. System 100 may include one or more memory storing devices (not shown in FIG. 1), a single board computer (SBC) 102 (referred herein as SBC 102) that may contain one or more processors 103 (referred herein as processor 103), a system board (SB) 104 (referred herein as SB 104), a high voltage (HV) module 106 (referred herein as HV module 106), and a low voltage (LV) module 108 (referred herein as LV module 108). Processor 103 and/or SBC 102 may control, manage, and/or collect data in system 100. Processor 103 may be an ASIC (Application Specific Integrated Circuit) or it may be a general purpose processor. Processor 103 may include more than one processor. For example, processors may be situated in parallel, series, or both in order to process all or part of the computer instructions that are to be processed. In one embodiment, Processor 103 may be in a dedicated circuit board independent of the SBC 102 and/or inside the SBC 102. SB 104, HV module 106, and LV module 108 may each include components. SB 104 may be connected to SBC 102 and the processor 103, HV module 106, and LV module 108. Processor 103 and/or SBC 102 may control, manage, and/or collect data from the HV module 106 and the LV module 108 including their components through their connection with SB 104. In one embodiment, processor 103 and/or SBC 102 may be connected to HV module 106 and its components and LV module 108 and its components. A power entry module (PEM) 110 (referred herein as PEM 110), which may be a further included component of system 100, may generate 24 Volts (24 V) direct current (DC) voltage rail to power the SBC 102, processor 103, the SB 104, the LV module 108, and the HV module 106. SB 104 may connect the PEM 110 to the SBC 102 and its components, the processor 103, the HV module 106 and its components, and the LV module 108 and its components. The processor 103 and/or SBC 102 may manage and/or control the voltage applied to components in the HV module 106 and/or the LV module 108. The SB 104 may gate the 24 V DC voltage rail from the PEM 110 using one or more DC relays (not shown in FIG. 1) to power components inside the SB 104 and inside the HV module 106. Similarly, the LV module 108 may contain one or more DC relays (not show in FIG. 1) to gate the 24 V DC voltage rail from the PEM 110 to power the components inside the SBC 102, processor 103, and LV module 108. In another embodiment, the SB 104 may include one or more DC relays to gate the 24 V DC voltage rail from the PEM 110 to power its components and the components in the HV module 106, the LV module 108, processor 103, and the SBC 102. In yet another embodiment, the PEM 110 may include one or more DC relays to gate the 24 V DC voltage rail to power the SBC 102 and its components, the processor 103, the SB 104 and its components, the HV module 106 and its components, and the LV module 108 and its components. In yet a further embodiment, the PEM 110 may be directly connected to one or more DC relays or power supplies to gate the 24 V DC voltage rail to power the SBC 102 and its components, the processor 103, SB 104 and its components, the HV module 106 and its components, and the LV module 108 and its components.

The HV module 106 may include a high voltage board 112 (referred herein as HV board 112), an isolated low voltage (LV) DC supply 114 (referred herein as isolated LV DC supply 114), a high voltage supply 116 (referred herein as HV supply 116), at least one capacitor bank 118 including one or more capacitors, high voltage switches 119 (referred herein as HV switches 119), a pulse modulator (PM) board 120 (referred herein as PM board 120), an instrument output 122, a processing assembly (PA) 124 (referred herein as PA 124), and a calibration port 126, which may all constitute components of the HV module 106.

The HV board 112 may gate the isolated LV DC supply 114 from the PEM 110 using a DC relay (not shown in FIG. 1) to supply the HV supply 116. The PEM 110 may supply power to the isolated LV DC supply 114 to power the HV supply 116. The HV board 112 may manage and/or regulate the required amount of power to the HV supply 116 based on the required demand of power directed by processor 103 and/or SBC 102. Processor 103 and/or SBC 102 may be connected to the HV board 112 through SB 104. The HV supply 116 may in turn charge the at least one capacitor bank 118 to maintain or quickly generate the required amount of voltage for electroporation. The required amount of voltage may range between −1000 Volts to +1000 Volts where the required amount of voltage may be +/−600 Volts, +/−700 Volts, +/−800 Volts, +/−900 Volts, +/−1000 Volts, and/or higher. The at least one capacitor bank 118 may contain one or more capacitor to generate the required amount of voltage. In one embodiment, the at least one capacitor bank 118 may include 4 capacitors capable of generating the required amount of voltage. In another embodiment, the at least one capacitor bank 118 may include one or a plurality of capacitors to generate the required amount of voltage.

The PM board 120 may generate a positive and/or negative pulsing profile or wave that may be executed by the HV switches 119 to the instrument output 122. The HV supply 116 may draw power from the LV DC supply 114 to charge the at least one capacitor bank 118 to apply to the voltage to the HV switches 119. The PM board 120 may generate positive and/or negative pulsing profile or wave to the HV switches 119. The positive and/or negative pulsing profile or wave from the SBC 102, the processor 103, the SB 104, and/or the PM board 120 may be based on cells in a cell a suspension of fluid contained in a chamber of the PA 124 that SBC 102 may have identified. The PM board 120 may execute or apply the pulse width wave generated by the SBC 102 and/or processor 103 amounting to, resulting in, and/or equaling the required amount of voltage to be applied to the instrument output 122 via the HV switches 119. In another embodiment, the PM board 120 may generate the pulse width wave. In yet another embodiment, processor 103 may generate the pulse width wave. In one embodiment, the PM board 120, the SBC 102, and/or the processor 103 may generate the pulse width wave. The voltage in the instrument output 122 may be applied to the chamber containing the cells in the cell suspension contained in the fluid sample in the PA 124. The pulse width wave may be the combination of the positive pulsing profile or wave and the negative pulsing profile or wave, which may amount to, result in, and/or equal to the required amount of voltage to be applied to the chamber containing the cells in the cell suspension from the fluid sample in the PA 124 via the HV switches 119 and the instrument output 122. In another embodiment, the PM board 120 may generate positive and negative pulsing profile or wave for the HV board 112, the SBC 102, processor 103, and/or PM 120 to execute amounting to, resulting in, and/or equaling to the required amount of voltage applied by the HV switches 119 to the chamber containing the cells in the cell suspension in the fluid sample in the PA 124 via the instrument output 122.

Processor 103 and/or SBC 102 may apply a protocol that may include a timing or length of time and the correct amplitude and profile for the pulse width wave. Processor 103 and/or SBC 102 may command, manage, and/or regulate the HV board 112, the HV switches 119, the PM board 120, and the instrument output 122. In one embodiment, processor 103 and/or SBC 102 may also detect that there may be no fluid in the chamber of the PA 124 or may also detect that there may be no PA assembly plugged to the instrument output 122.

The instrument output 122 may be configured to provide two modes of operation: static electroporation and flow electroporation. The instrument output 122 may have a knob or other device that an operator or user may use to set whether system 100 may operate as static electroporation (with small scale) or flow electroporation (large scale). Processor 103 and/or SBC 102 may detect the position of the knob or other device, based on a user's input, on the instrument output 122 to command or instruct the HV module 106 to generate a protocol with the correct timing or length of time and the correct amplitude and profile for the pulse width wave. The static electroporation configuration may set the instrument output 122 to interface with a single PA 124 through a male/female “T-slot” where only a single or a plurality of cells in the cell suspension from the fluid samples (multi-wells) in the chamber of PA 124 may be electroporated for a period of time depending on the concentration of cells in the cell suspension in the volume of fluid samples. The flow electroporation configuration may set the instrument output 122 to interface with a single PA 124 through a pair of diagonally opposed male/female banana plugs where multiple fluid samples or wells containing cells in cell suspensions in the PA 124 may automatically and/or autonomously be electroporated such that processor 103 and/or SBC 102 may command the HV module 106 to run a protocol with one or more burst—each of some of amount of microseconds—separated by pause of time for the transfer of fluid for the next sample of fluid inside the PA 124. The protocol with one or more microseconds bursts separated by pause of time may be the pulse width wave. Flow electroporation may be a faster and efficient way to transfect a large volume of cells in a cell suspension in a fluid equal to 1 L in no more than 30 minutes.

A user may connect a meter device to calibration port 126 to cause processor 103 and/or SBC 102 to simulate a desired pulse width wave from the meter device where the HV board 112 may command the HV supply 116 to charge the at least one capacitor bank 118 to generate the required amount of voltage in order for the PM board 120 to generate a pulse width wave that the HV board 112 would execute in the instrument output 122 via the HV switches 119 based on the pulse width wave from the user's meter device although there may not be any PA 124 interfacing with the instrument output 122. The calibration port 126 may be used to calibrate system 100 once or periodically before the operation of the electroporation apparatus. In one embodiment, processor 103 and/or SBC 102 may be connected to the PM board 120 via the SB 104. In another embodiment, processor 103 and/or SBC 102 may be connected to the PM board 120 via the connection of the SB 104 to the HV board 112. In yet another embodiment, processor 103 and/or SBC 102 may be connected to the PM board 120 via the connection of the SB 104 to the HV board 112 and the HV switches 119. In another embodiment, processor 103 and/or SBC 102 may be connected to the HV board 112, the HV board 112, the at least one capacitor bank 118, the HV switches 119, the PM board 120, the instrument output 122, the PA 124, the calibration port 126 via the connection of the SB 104 to the HV module 106.

The LV module 108 may include an isolated LV DC supply 144, a display and touch screen 146 or user interface, a barcode reader 148, one or more fans 150 (referred herein as fan 150), one or more speakers 152 (referred herein as speaker 152), a power button 154, one or more universal serial bus 3.0 ports 156 (referred herein as USB 3.0 ports 156), a light emitting diode (LED) board 158 (referred herein as LED board 158), an entry pump 160, an entry valve 162, an exit pump 164, and an exit valve 166, which may all constitute components of the LV module 108.

The SB 104 may gate the isolated LV DC supply 114 from the PEM 110 using one or more DC relays (not shown in FIG. 1). In another embodiment, isolated LV DC supply 114 may gate the PEM 110 using one or more DC relays (not shown in FIG. 1) from the LV module 108. In one embodiment, processor 103 and/or SBC 102 may manage, control and/or collect data from the isolated LV DC supply 144, the display and touch screen 146, the barcode reader 148, the fan 150, the speaker 152, the power button 154, the USB 3.0 ports 156, the LED board 158, the entry pump 160, the entry valve 162, the exit pump 164, and the exit valve 166 via the connection of the SB 104. In another embodiment, the SB 104 may be connected to all of the components in the LV module 108, and processor 103 and/or SBC 102 may manage and/or control the isolated LV DC supply 114, the display and touch screen 146, the barcode reader 148, the fan 150, the speaker 152, the power button 154, the USB 3.0 ports 156, the LED board 158, the entry pump 160, the entry valve 162, the exit pump 164, and the exit valve 166.

In the example embodiment, a display and touch screen 146 is used.

However, other embodiments may alternatively be implemented other known user interface devices. For example, display and touch screen 149 may instead be implemented using a non-touch screen display and an alternative means of user input, such as a keyboard, a mouse, programable soft keys, hard coded buttons, or the like.

Processor 103 and/or SBC 102 may collect transfection data associated with the cells in the cell suspension from the fluid sample inside the chamber of PA 124 for storage in the one or more storing devices in system 100 (not shown in FIG. 1). Processor 103 and/or SBC 102 may allow an operator or user to manipulate the data on the display and touch screen 146. The SBC 102 may provide system 100 with a computer functionality to operators or users where there may not be a need for a standalone desktop or laptop computer. The SBC 102 may command, manage, and/or regulate the display and touch screen 146, the barcode reader 148, the fan 150, the speaker 152, the power button 154, the USB 3.0 ports 156, the LED board 158, the entry pump 160, the entry valve 162, the exit pump 164, and the exit valve 166. An operator or user may scan a PA 124 containing a fluid sample to barcode reader 148 for processor 103 and/or SBC 102 to identify and associate the required pulse width wave profile to apply to the cells in the cell suspension from the fluid sample based on a protocol selected by the operator or user. The scanned information that may be read by barcode reader 148 may be stored in the one or more storing devices for later identification and association with results in the SBC 102 and/or manipulated data generated by an operator or user on the display and touch screen 146. Processor 103 and/or SBC 102 may receive one or more commands from an operator or user via the display and touch screen 146 to execute a specific required pulse width wave profile or protocol to apply via the instrument output 122 to the cells in the cell suspension from the fluid sample.

The processor 103 and/or SBC 102 may activate fan 150 to cool the components in the LV module 108. The processor 103 and/or SBC 102 may use speaker 152 to communicate results to operators or users based on inputs from display and touch screen 146. The processor 103 and/or SBC 102 may detect via the SB 104 that an operator or user may have pressed the power button 154 so that processor 103 and/or SBC 102 may power up system 100. An operator or user may store saved results from SBC 102 and/or manipulated data generated on display and touch screen 146 in the one or more storing devices to transfer to an external storing device via the USB 3.0 ports 156. The power button 154 may be illuminated by one or more colors, such as blue, yellow, red, green, or orange, via processor 103 and/or SBC 102 using LED board 158 where a first color may indicate that the system 100 is “on,” and a second color may indicate that the system 100 is “off.” Furthermore, the processor 103 and/or SBC 102 may use the LED board 158 to illuminate a hollow rectangular shape section on the periphery of the instrument output 122 with one or more colors, such as blue, yellow, red, green, or orange. In another embodiment, the processor 103 and/or SBC 102 may illuminate the hollow rectangular shape section on the periphery of the instrument output 122 with one or more colors, such as blue, yellow, red, green, or orange. The color of the hollow rectangular shape section around the periphery of the instrument output 122 may indicate to the operator or user the progress or status of the electroporation process. Moreover, processor 103 and/or SBC 102 may change the color of the hollow rectangular shape section on the periphery of the instrument output 122.

Processor 103 and/or SBC 102 may determine the type of fluid and the viscosity of the fluid in the chamber of PA 124 where a high number of cells in a cell suspension may indicate a high level of viscosity in the fluid, which may require a high amount of pressure to move the fluid inside the chamber of the PA 124 during flow electroporation. Processor 103 and/or SBC 102 may directly use and/or command the use of entry pump 160 and exit pump 164. Entry pump 160 and exit pump 164 may be used by circulating air around the chamber of the PA 124 to create positive or negative air pressure to move or draw the fluid sample into and out of the chamber of PA 124. For example, the entry pump 160 may be used to create negative air pressure or vice versa by sucking air or vice versa out of the chamber in PA 124, which may in turn draw the fluid sample from a filled 1 L bag into the chamber in PA 124. After the electroporation process may be executed, the exit pump 164 may generate positive air pressure or vice versa by blowing air or vice versa out of the chamber in PA 124, which may in turn draw the fluid sample from the chamber in PA 124 to an empty or a filling 1 L bag containing the transfected cells in the cell suspension in the fluid sample.

Processor 103 and/or SBC 102 may use entry valve 162 or exit valve 166. Processor 103 and/or SBC 102 may control with precision the amount of fluid that circulates from the filled 1 L bag into the chamber in PA 124 and out to the empty or filling 1 L bag based on processor 103 and/or SBC 102 identifying the type and viscosity of the fluid. For example, in accord with the entry pump 160 and exit pump 164, processor 103 and/or SBC 102 may actuate the entry valve 162 and exit valve 166 to stop the fluid from flowing from the filled 1 L bag to the chamber of PA 124 to the empty or filling 1 L bag even if entry pump 160 and/or exit pump 164 may be exerting large positive and/or negative pressures because entry valve 162 and exit valve 166 may squeeze one or more tubes tied to the PA 124 for fluid transfer prevention. Processor 103 and/or SBC 102 may accelerate or slow down the flow of fluid from the filled 1 L bag to the chamber of PA 124 to the empty or filling 1 L bag even if entry pump 160 and/or exit pump 164 may be exerting low levels positive and/or negative pressures because entry valve 162 and exit valve 166 may squeeze or release one or more tubes tied to the PA 124 for fluid transfer deceleration or acceleration.

An operator or user may temporarily press emergency stop (E-stop) button 168 (referred herein as E-stop button 168) to immediately stop the electroporation process or the pulse width wave that may be generated by the PM board 120 via the HV switches 119 and/or executed by the HV board 112 where a D flip-flop (not shown in FIG. 1) in SB 104 may override, disable, block, and/or bypass processor 103 and/or SBC 102 and directly shut or terminate the HV board 112, the HV switches 119. and the PM board 120 in the HV module 106. The D flip-flop may override, disable, block, and/or bypass processor 103 and/or SBC 102 to manage and/or control the HV module 106 and its components, and the D flip-flop may still utilize processor 103 and/or SBC 102 to manage and/or control the LV module 108 and its components. Furthermore, the D flip-flop in SB 104 may communicate with processor 103 and/or the SBC 102 via the USB 3.0 ports of the SBC 102 to display a status on the display and touch screen 146 that the E-stop button 168 may have been pressed by the operator and user. The E-stop 168 may override, disable, block, and/or bypass processor 103 and/or SBC 102 for immediate termination of any electroporation in the HV module 106. Furthermore, the E-stop 168 may be implemented to stop large voltages during the electroporation process for emergency situations, where near instantaneous deactivation may be advantageous. For example, the E-stop 168 may be used to prevent the destruction of cells in the cell suspension from the fluid samples, to prevent system 100 from being destroyed, to prevent any leaked fluid from electrocuting an operator or user, or to prevent the explosion of a flammable fluid sample. Moreover, the E-stop 168 and the D flip-flop in SB 104 may not depend on the software on system 100 to execute the immediate termination of the electroporation process in the HV module 106 when overriding, disabling, blocking, and/or bypassing processor 103 and/or SBC 102. The E-stop button 168 may be illuminated with a red LED light by processor 103 and/or SBC 102.

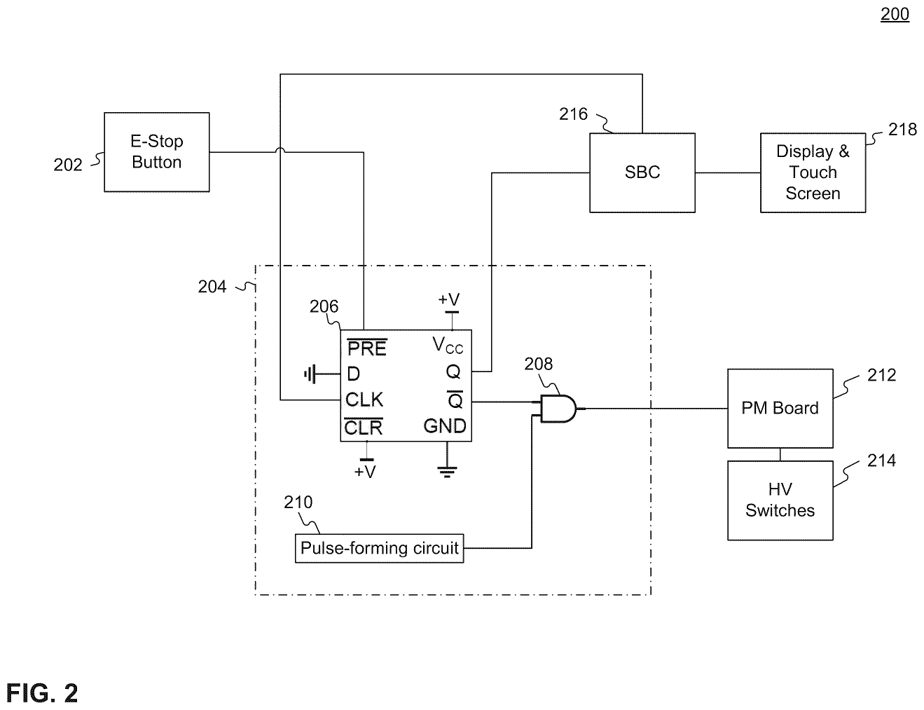

FIG. 2 is a schematic block diagram illustrating an exemplary non software-based emergency stop feature, consistent with the disclosed embodiments. System 200 may include E-stop button 202 (also referring to the E-stop button 168 in FIG. 1)—E-stop button 168 of FIG. 1 may be the same as E-stop button 202 of FIG. 2—, SB 204 (also referring to the SB 104 in FIG. 1), D flip-flop 206, Gate 208, Pulse-forming circuit 210, PM board 212 (also referring to the PM board 120 in FIG. 1), HV switches 2144 (also referring to the HV switches 119 in FIG. 1), SBC 216 (also referring to the SBC 102 in FIG. 1), and display and touch screen 218 (also referring to the display and touch screen 146 in FIG. 1), which may all constitute components of system 200. System 200 may be subcomponents of system 100 in FIG. 1. The SB 204, D flip-flop 206, Gate 208, and/or the pulse-forming circuit 210 may be an electronic circuit.

The E-stop button 202 is connected to D flip-flop 206. The D flip-flop 206 maybe located inside SB 204. The D flip-flop 206 may contain a preset input pin (P̅R̅E̅), input pin (D), clock input pin (CLK), clear input pin (C̅L̅R̅), supply pin (V_{CC}), output pin (Q), inverted output pin (Q̅), and ground pin (GND). To avoid relying on the clocking feature of the D flip-flop 206, the input pin (D) may be connected to ground such that the clock input pin's (CLK) clock rising edge functionality may be used to reset the D flip-flop 206, the ground pin (GND) may be connected to ground, the supply pin (V_{CC}) may be connected to a voltage level (+V) required to operate the D flip-flop 206, and the clear input pin (C̅L̅R̅) may also be connected to a logically high voltage level and thus disabled.

Furthermore, the E-stop button 202 may be connected to the preset input pin (P̅R̅E̅) of the D flip-flop 206 such that when a user may momentarily press on the E-stop button 202, a momentary signal may be latched—the momentary signal may be held, saved, and/or preserved by the D flip-flop 206—by the preset input pin (P̅R̅E̅) where a preset input signal may be of a logical value of “1.” In other embodiments, the preset input signal may be a low voltage or ground connection. The preset input pin (P̅R̅E̅) may be asynchronous and may react immediately when the preset input signal may be pulled low—assigned a logical value of “0”—or may be pulled high—assigned a logical value of “1”—, unlike the traditional input pin (D) —not connected to ground but to an input signal—which may only react on a rising edge from a clock signal on the clock input pin (CLK). Preset input pin (P̅R̅E̅) may be assigned the logical value of “1,” even if the operator or user may subsequently press the E-stop button 202 where system 200 may latch on to the signal received from the E-stop button 202.

In some embodiments, it may be advantageous for system 200 to output the latched signal via two separate outputs where the first output may be a hardware safety mechanism, and the second output (also referring to a logical signal) may be a software safety mechanism. The latched signal—second output—for the software safety mechanism may be outputted directly to output pin (Q). The latched signal—first output—for the hardware safety mechanism may be outputted directly to inverted output pin (Q̅). In the hardware safety mechanism, system 200 may only rely on the D flip-flop 206 to directly deactivate power in the HV module 106 (shown in FIG. 1) where the D flip-flop 206 may also override, disable, block, and/or bypass the logical signal from processor 103 and/or SBC 216 via the SB 204. An advantage to the hardware safety mechanism may be that no software commands and/or logical signal may be relied upon, which may be susceptible to timing or logical errors—software delays—in processor 103 and/or SBC 216 that may prevent immediate deactivation of power—electroporation—in the HV module 106, where the combination of the positive pulsing profile or wave and the negative pulsing profile or wave amounting to, resulting in, and/or equaling to the required amount of voltage may immediately not be applied to the chamber containing the cells in the cell suspension from the fluid sample in the PA 124 via the chain of the PM board 212, the HV switches 214, and the instrument output 122 (shown in FIG. 1). In the software safety mechanism, system 200 may rely on both processor 103 and/or SBC 216 and the D flip-flop 206 where the D flip-flop 206 may send the latched signal to processor 103 and/or SBC 216 to display a message to the operator or user via the display and touch screen 218 that the electroporation process may have been terminated, or the D flip-flop 206 may receive an input from an operator or user via the processor 103, controlled by the SBC 216, from the display and touch screen 218 requesting reactivation of the electroporation process. In one embodiment, the D flip-flop 206 may receive an input from an operator or user via the SBC 216 from the display and touch screen 218 requesting reactivation of the electroporation process. In another embodiment, the D flip-flop 206 may receive an input from an operator or user via the processor 103 from the display and touch screen 218 requesting reactivation of the electroporation process. The software safety mechanism may be susceptible to timing or logical errors—software delays—in the logical signal of processor 103 and/or SBC 216 for the operation of components in the LV module 108 and/or HV module 106, but those software delays may not inadvertently affect the D flip-flop 206 from directly deactivating, overriding, disabling, blocking, and/or bypassing power for electroporation in the HV module 106.

The latched signal from the preset input pin (P̅R̅E̅) may be directly outputted to output pin (Q) as the logical value of “1,” which may be communicated to the SBC board 216 where processor 103 may cause the display and touch screen 218 to display a message to the operator or user that the E-stop feature may be active. The latched signal from preset input pin (P̅R̅E̅) may be directly outputted to inverted output pin (Q̅) as logical value “0,” which may be sent to the HV module 106 and/or PM board 212 and the HV switches 214 to stop any execution and/or generation of pulse width wave at the instrument output 122 where the D flip-flop 206 may directly deactivate, override, disable, block, and/or bypass processor 103 and/or SBC 216 from managing and/or controlling the HV module 106 and its components in FIG. 1. In another embodiment, a gate 208 may have as a first input the latched signal from preset input pin (P̅R̅E̅) directly outputted to inverted output pin (Q̅) as logical value “0,” and a second input of “1” from a pulse-forming circuit 210. The first input of “0” coupled with the second input of “1” may cause the gate 208 to output the logical value of “0” to the PM board 212. The output logical value of “0” from the gate 208 to the PM board 212 may disable or stop the HV switches 214 from applying the generated positive and/or negative pulsing profile or wave from the PM board 212 into the instrument output 122 of FIG. 1. In yet another embodiment, the output logical value of “0” from the gate 208 to the PM board 212 may disable or stop the PM board 212 from generating a positive and/or negative pulsing provide or wave, and the HV switches 214 may not apply any voltage to the instrument output 122.

The display of the message on the display and touch screen 218 may request for the user to reset the latched signal from the E-stop 202 back to “0” by pressing a graphical user interface (GUI) icon on the display and touch screen 218. The logical value of “1” from the user requesting to reset the E-stop button 202's signal may be sent to the clock input pin (CLK), which coupled with the input pin (D) being always set to the logical value of “0” may reset the preset input pin (P̅R̅E̅) to “0.” In one embodiment, the signal from the user requesting to reset the E-stop button 202 may be received by the display and touch screen 218, may be sent to the SBC 216 and/or processor 103, and may be sent to the clock input pin (CLK) from the SBC 216 and/or processor 103. With the preset input pin (P̅R̅E̅) set reset to the logical value of “0,” the output pin (Q) may have a logical value of “0,” which may indicate to the SBC 216 and/or processor 103 that no E-stop button 202 may have been pressed, and normal operation may proceed as described in FIG. 1. Furthermore, inverted output pin (Q̅) in the D flip-flop 206 may have the logical value of “1” in response to the preset input pin (P̅R̅E̅) reset to the logical value of “0.” The inverted output pin (Q̅) having a logical value of “1” may allow processor 103 and/or SBC 216 to command the PM board 212 to generate a pulse width wave executed by the HV switches 214 where the D flip-flop 206 may no longer deactivate, override, disable, block, and/or bypass processor 103 and/or SBC 216 due to the user requesting to reset the E-stop button 202. In another embodiment, the gate 208 may receive from its first input the logical value of “1” from the inverted output pin (Q̅) in normal operation, and the gate 208 may receive from its second input the logical value of “1” from the pulse-forming circuit 210, which may cause gate 208 to output the logical value of “1” to the PM board 212. With the output being the logical value of “1” from the gate 208, the PM board 212 and the HV switches 214 may resume their normal operation where processor 103 and/or SBC 216 may command the PM board 212 to generate a pulse width wave executed by the HV switches 214 where the D flip-flop 206 may no longer override, disable, block, and/or bypass processor 103 and/or SBC 216 due to the user requesting to reset the E-stop button 202.

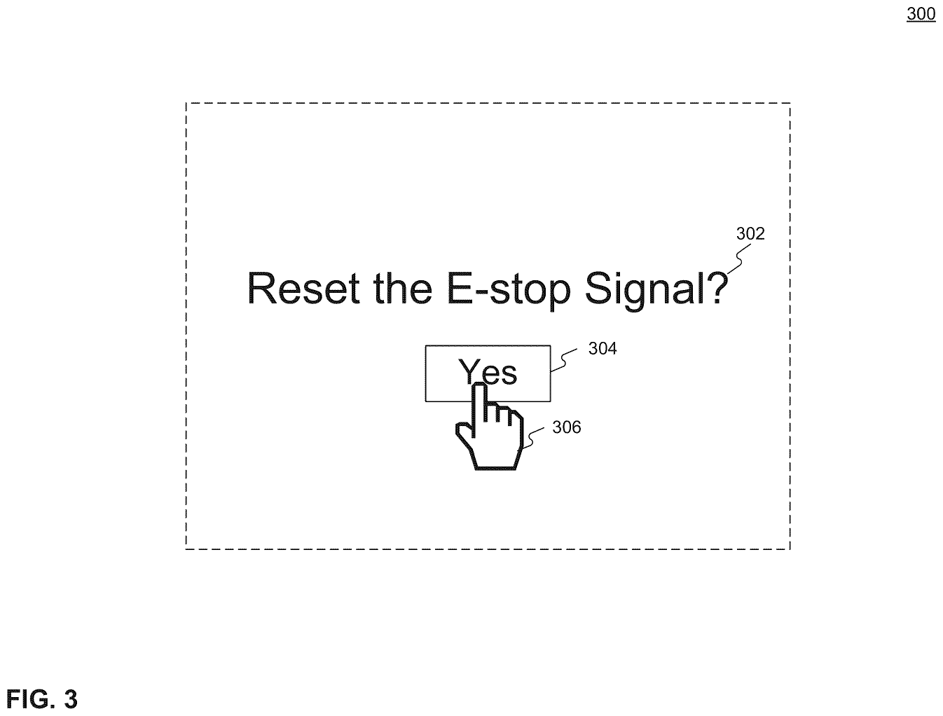

FIG. 3 depicts an illustration of a graphical user interface of the display & touch screen for resetting the emergency stop signal, consistent with the disclosed embodiments. GUI 300 on display and touch screen 218 of FIG. 2 (also referring to display and touch screen 146 of FIG. 1) may display message 302 reading “Reset the E-stop Signal?.” The operator or user may choose to touch or press on GUI icon 304, which when the user may touch or press 306 on GUI icon 304, may cause the E-stop 202 to be reset back to “0.” The logical value of “1” from the user requesting to reset the E-stop button 202's signal may be sent to the clock input pin (CLK), which coupled with the input pin (D) being always set to the logical value of “0” or ground, may reset the preset input pin (P̅R̅E̅) to “0.”

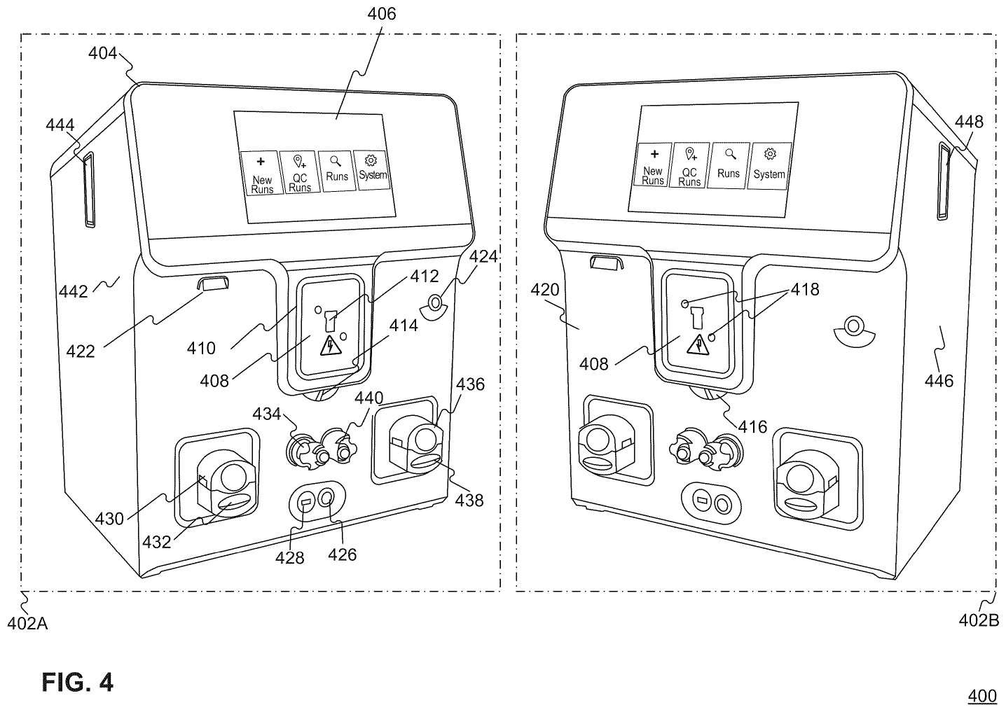

FIG. 4 is an exemplary illustration of the integrated electroporation system and its components in the static electroporation and flow electroporation configurations, consistent with the disclosed embodiments. FIG. 4 includes view 402A and view 402B of system 400 (also referring to the system 100 and system 200). In view 402A, system 400 may have a bezel subassembly 404 having a “T” shaped form with a thick and long horizontal section and a thin and short vertical section. The bezel subassembly 404 may include the display and touch screen 406 (also referring to the display and touch screen 146 in FIGS. 1 and 218 in FIG. 2) at the center of its thick and long horizontal section and an instrument output 408 (also referring to the Instrument output 122 in FIG. 1) at the center of the thin and short vertical section. The Instrument output 408 may be surrounded by a hollow rectangular shape section 410 on its periphery that may be illuminated by LED board 158 from FIG. 1. The instrument output 408 in view 402A may be configured for static electroporation where a female “T-slot” 412 on the instrument output 408 may interface with the male “T-slot” (not shown in FIG. 4) on the PA 124 in FIG. 1. The female “T-slot” 412 may be illuminated to indicate to an operator or user that the static electroporation configuration may be active. The knob 414 may be embedded below the instrument output 408 and the thin and short vertical section of bezel subassembly 404. The knob 414 may be positioned by the operator or user by rotating the knob 414 clockwise to engage the static electroporation configuration. In view 402B, the instrument output 408 may have its knob 416 rotated counter-clockwise by the operator or user to engage the flow electroporation configuration where a pair of diagonally opposed female banana plugs 418 on the instrument output 408 may interface with a pair of diagonally opposed male banana plugs (not shown in FIG. 4) on the PA 124 in FIG. 1. The pair of diagonally opposed female banana plug 418 may be illuminated to indicate to an operator or user that the flow electroporation configuration may be active.

In view 402B of FIG. 4, system 400 may have a lower fascia subassembly 420 interfacing a lower periphery edges or profile of the bezel subsassembly 404. The lower fascia subassembly 420 may include, from view 402A, a barcode reader 422 (also referring to the barcode reader 148 of FIG. 1), an E-stop button 424 (also referring to the E-stop button 168 of FIGS. 1 and 202 of FIG. 2), a power button 426 (also referring to the power button 154 of FIG. 1), an USB 3.0 port 428 (also referring to the USB 3.0 ports 156 of FIG. 1), an entry pump 430 (also referring to the entry pump 160 of FIG. 1) having a knob 432 to attach a tube of the PA 124 in the flow electroporation configuration, an entry valve 434 (also referring to the entry valve 162 of FIG. 1), an exit pump 436 (also referring to the exit pump 164 of FIG. 1) having a knob 438 to attach a tube of the PA 124 in the flow electroporation configuration, and an exit valve 440 (also referring to the exit valve 166 of FIG. 1).

In view 402A of FIG. 4, system 400 may have a left side panel 442 interfacing the left side edges or profile of the thick and long section of the bezel subassembly 404 and the lower fascia subassembly 420. The left side panel 442 may include a left side hook subassembly 444 in a stowed configuration. In view 402B of FIG. 4, system 400 may have a right-side panel 446 interfacing the right side edges or profile of the thick and long section of the bezel subassembly 404 and the lower fascia subassembly 420. The right-side panel 448 may include a right side hook subassembly 448 in a stowed configuration.

E-stop button 424 in view 402A may be located in the top right corner of the lower fascia subassembly 420 in view 402B, in line with the female “T-slot” 412. E-stop button 424 may be located in other embodiments in a similar location, such as an equivalent location on the left side corner of the lower fascia subassembly 420. Placement of the E-stop button 424 in this or a similar location may have certain advantages. For example, the placement of the E-stop button 424 may allow easy access without disrupting one or more tubes of the PA 124, and away from any potential leaks out of the tubing and/or the PA 124. The placement may also place or situate the E-stop button 424 closer to the operator or user's eye level, making it easier to identify the E-stop button 424 in an emergency.

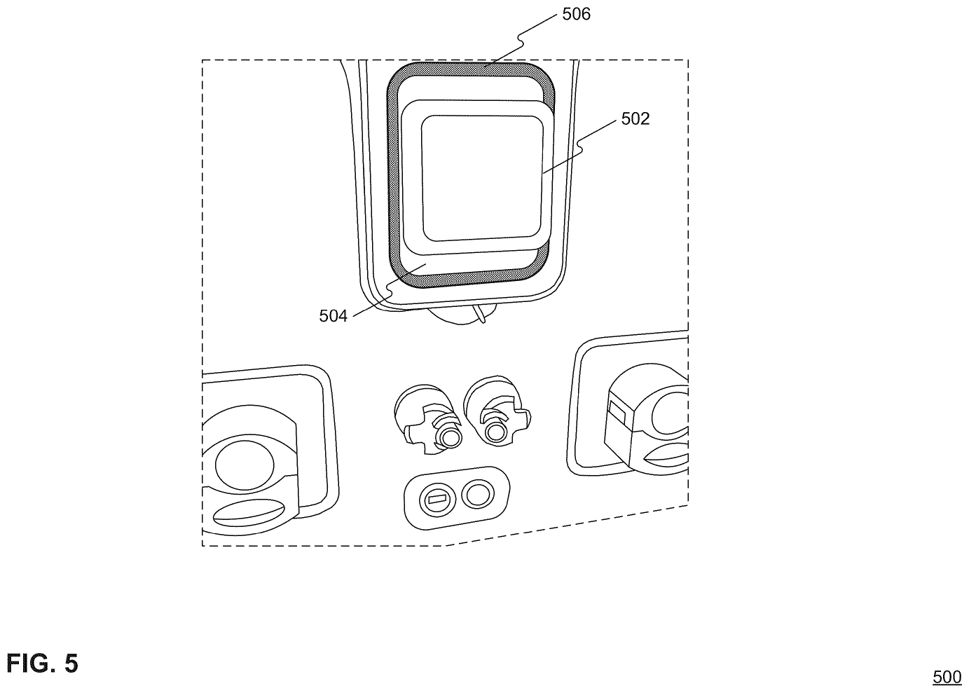

FIG. 5 is an exemplary illustration of a processing assembly interfacing an instrument output in the static electroporation configuration, consistent with the disclosed embodiments. FIG. 5 shows a close up view of system 500 (also referring to the system 100 of FIGS. 1, 200 of FIG. 2, and 400 of FIG. 4) where a PA 502 (also referring to the PA 124 of FIG. 1) may be configured for static electroporation. PA 502 may interface with instrument output 504 (also referring to the instrument output 122 of FIGS. 1 and 408 of FIG. 4) where the female “T-slot” 412 in FIG. 4 may interface with the male “T-slot” of the instrument output 504 (not shown in FIG. 5). A hollow rectangular shape section 506 (also referring to the hollow rectangular shape section 410 of FIG. 4) on the periphery of the instrument output 504 may be illuminated by LED board 158 from FIG. 1 to indicate to the operator or user that system 500 may be in the static electroporation configuration.

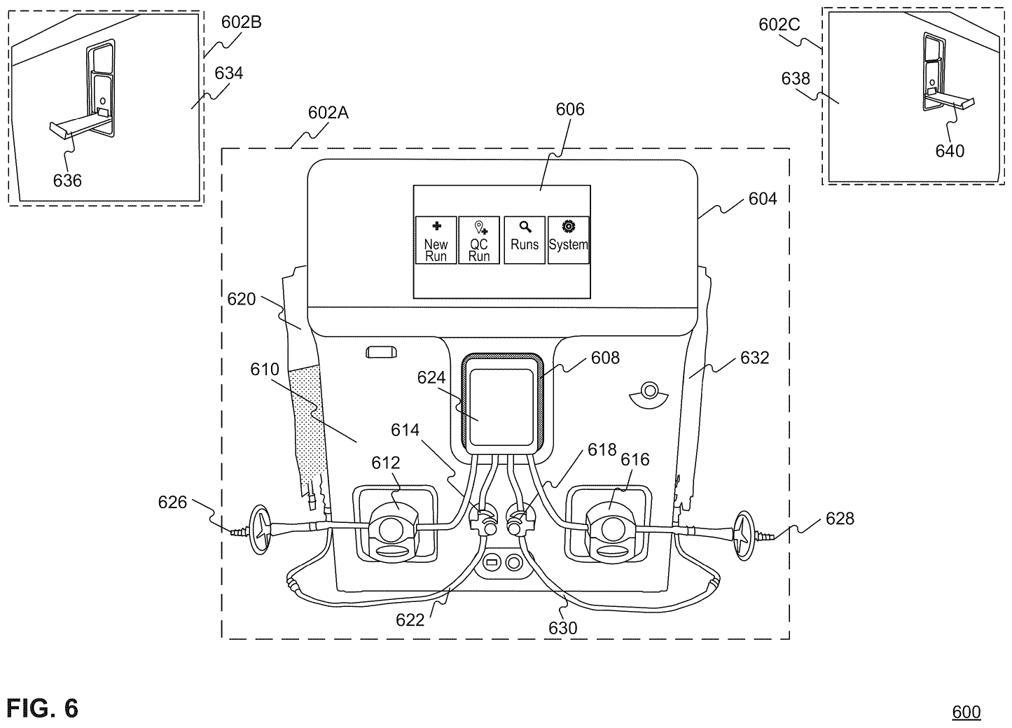

FIG. 6 is an exemplary illustration of a processing assembly interfacing an instrument output in the flow electroporation configuration, consistent with the disclosed embodiments. FIG. 6 shows system 600 (also referring to the system 100 of FIGS. 1, 200 of FIGS. 2, 400 of FIG. 4, and 500 of FIG. 5) with view 602A, view 602B, and view 602C. View 602A of FIG. 6 includes a bezel subassembly 604 (also referring to the bezel subassembly 404 of FIG. 4) having the display and touch screen 606 (also referring to the display and touch screen 146 of FIGS. 1, 218 of FIG. 2, and 406 of FIG. 4) and a hollow rectangular shape section 608 (also referring to the hollow rectangular shape section 410 of FIGS. 4 and 506 of FIG. 5) on the periphery of the instrument output 504 (not shown in FIG. 6).

View 602A in FIG. 6 shows a lower fascia subassembly 610 (also referring to the lower fascia subassembly 420 of FIG. 4) including an entry pump 612 (also referring to the entry pump 160 of FIGS. 1 and 430 of FIG. 4), an entry valve 614 (also referring to the entry valve 162 of FIGS. 1 and 434 of FIG. 4), an exit pump 616 (also referring to the exit pump 164 of FIGS. 1 and 436 of FIG. 4), and an exit valve 618 (also referring to the exit valve 166 of FIGS. 1 and 440 of FIG. 4).

In one embodiment, a PA configured for flow electroporation may include a filled 1 L bag 620, an entry tube 622, a chamber 624, an inlet/outlet tube 626, an inlet/outlet tube 628, an exit tube 630, and an empty or filling 1 L bag 632. The filled 1 L bag 620 may be connected to the entry tube 622, which may be connected to the entry valve 614. The entry tube 622 may enter the chamber 624, and the inlet/outlet tube 626 may exit the chamber 624 and may be connected to the entry pump 612. The inlet/outlet tube 626 may exit the entry pump 612, and the inlet/outlet tube's 626 end orifice may be exposed to air.

Furthermore, an inlet/outlet tube 628 may exit the chamber 624 and may be connected to the exit pump 616. The inlet/outlet 628 may exit the exit pump 616, and the inlet/outlet's 628 end orifice may be exposed to air. Moreover, the exit tube 630 may exit the chamber 624 and be connected to the exit valve 618. The exit tube 630 may exit the exit valve 618 to connect to the empty or filling 1 L bag 632.

During operation, entry pump 612 may pressurize the inlet/outlet tube 626, the chamber 624, and the entry tube 622 to blow air out of the inlet/outlet tube 626's end orifice, which may cause fluid from the filled 1 L bag 620 to flow through entry tube 622—regulated by valve 614—and into the chamber 624. Similarly, exit pump 612 may pressurize the inlet/outlet tube 628, the chamber 624, and the exit tube 630 to blow air out of the inlet/outlet tube 628's end orifice, which may cause fluid inside the chamber 624 to flow through exit tube 630—regulated by valve 618—and into the empty or filing 1 L bag 632. The fluid in the chamber 624 may electroporated by the instrument output 504. The pair of diagonally opposed male banana plugs (not shown in FIG. 6) from chamber 624 may interface with the pair of diagonally opposed female banana plugs 418 (not shown in FIG. 6) on the instrument output 504. SB 204 may manage and coordinate the flow of fluid in the chamber 624 by controlling entry pump 612, entry valve 614, exit pump 616, and exit valve 618. For example, SB 204 may cause entry pump 612 and exit pump 616 to absorb air through inlet/outlet tube 626's end orifice and inlet/outlet tube 628's end orifice, respectively, to prevent the fluid from entering or exiting the chamber 624 during electroporation where entry valve 614 may prevent the flow of fluid in entry tube 622, and exit valve 618 may prevent the flow of fluid in exit tube 630. SB 204 may determine the correct amount of fluid in chamber 624 for electroporation via current sensor 134.

View 602B in FIG. 6 shows the left side panel 634 (also referring to left side panel 442 of FIG. 4) with a left side hook subassembly 636 (also referring to left side hook assembly 444 of FIG. 4) in a deployed configuration where the left side hook subassembly 636 may support the filled 1 L bag 620 in view 602A. View 602C in FIG. 6 shows the right side panel 638 (also referring to left side panel 446 of FIG. 4) with a right side hook subassembly 640 (also referring to left side hook assembly 448 of FIG. 4) in a deployed configuration where the right side hook subassembly 640 may support the empty or filling 1 L bag 632 in view 602A.

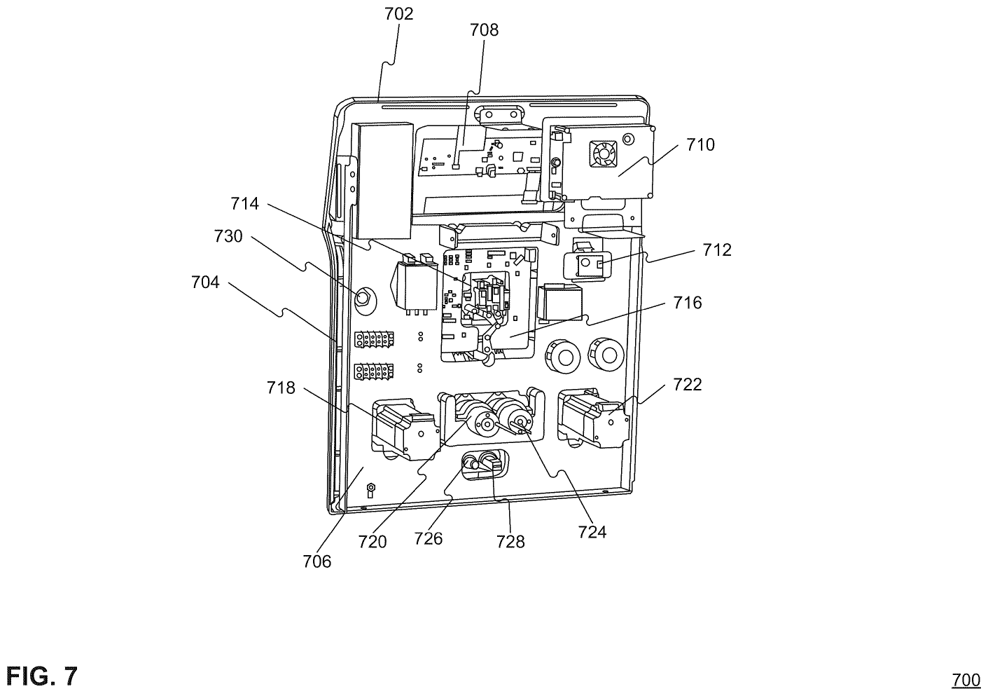

FIG. 7 is an exemplary illustration of the electroporation system with the packaging of low voltage components and the display and touch screen, consistent with the disclosed embodiments. FIG. 7 displays subsystem 700 having the LV module 108 of FIG. 1 and the SB 104 of FIG. 1. Subsystem 700 may include a bezel subassembly 702 (also referring to the bezel subassembly 404 of FIGS. 4 and 606 of FIG. 6), a lower fascia subassembly 704 (also referring to the lower fascia subassembly 420 of FIGS. 4 and 610 of FIG. 6), a front panel subassembly 706, a display and touch screen 708 (also referring to the display and touch screen 146 of FIGS. 1, 214 of FIGS. 2, 406 of FIG. 4, and 606 of FIG. 6), a SBC 710 (also referring to the SBC 102 of FIGS. 1 and 212 of FIG. 2), a barcode reader 712 (also referring to the barcode reader 148 of FIGS. 1 and 422 of FIG. 4), an Instrument output 730 (also referring to the Instrument output 122 of FIGS. 1, 408 of FIGS. 4, 504 of FIG. 5), an LED board 716 (also referring to the LED board 158 of FIG. 1), an exit pump 718 (also referring to the exit pump 164 of FIGS. 1, 436 of FIG. 4, and 616 of FIG. 6), the exit valve 720 (also referring to the exit valve 166 of FIGS. 1, 440 of FIG. 4, and 618 of FIG. 6), an entry pump 722 (also referring to the entry pump 160 of FIGS. 1, 430 of FIG. 4, and 612 of FIG. 6), an entry valve 724 (also referring to the entry valve 162 of FIGS. 1, 434 of FIG. 4, and 614 of FIG. 6), a power button 726 (also referring to the power button 154 of FIGS. 1 and 426 of FIG. 4), an USB 3.0 port 728 (also referring to the USB 3.0 ports 156 of FIGS. 1 and 428 of FIG. 4), and an E-stop 730 (also referring to the E-stop button 168 of FIGS. 1, 202 of FIG. 2, and 424 of FIG. 4).

Facing the front panel subassembly as shown in FIG. 7, the bezel subassembly 702 and the lower fascia subassembly 704 may be mounted on the front panel subassembly 706. The display and touch screen 708 may be mounted to the bezel subassembly 702. The SBC 710 may be mounted on the top right side of the front panel subassembly 706. The barcode reader 712 may be mounted to the top right of the lower fascia subassembly 704 and may be mounted beneath the SBC 710 on the front panel subassembly 706. The instrument output 730 may be mounted on the bezel subassembly 702. The LED board 716 may be mounted on the instrument output 730 facing the front panel subassembly 706. The exit pump 718 may be mounted on at the bottom left of the lower fascia subassembly 704 and the bottom left of the front panel subassembly 706. The exit valve 720 may be mounted at the center of the lower fascia subassembly 704 to the right of the exit pump 718. The entry pump 722 may be mounted on at the bottom right of the lower fascia subassembly 704 and the bottom right of the front panel subassembly 706. The entry valve 724 may be mounted at the center of the lower fascia subassembly 704 to the left of the entry pump 722 and to the right of the exit valve 720. The power button 726 may be mounted at the bottom center of the lower fascia subassembly beneath the exit valve 720. The USB 3.0 port 728 may be mounted at the center of the lower fascia subassembly 704 beneath the entry valve 724 and to the right of the power button 726. The E-stop button 730 may be mounted to the top left of the fascia subassembly 704.

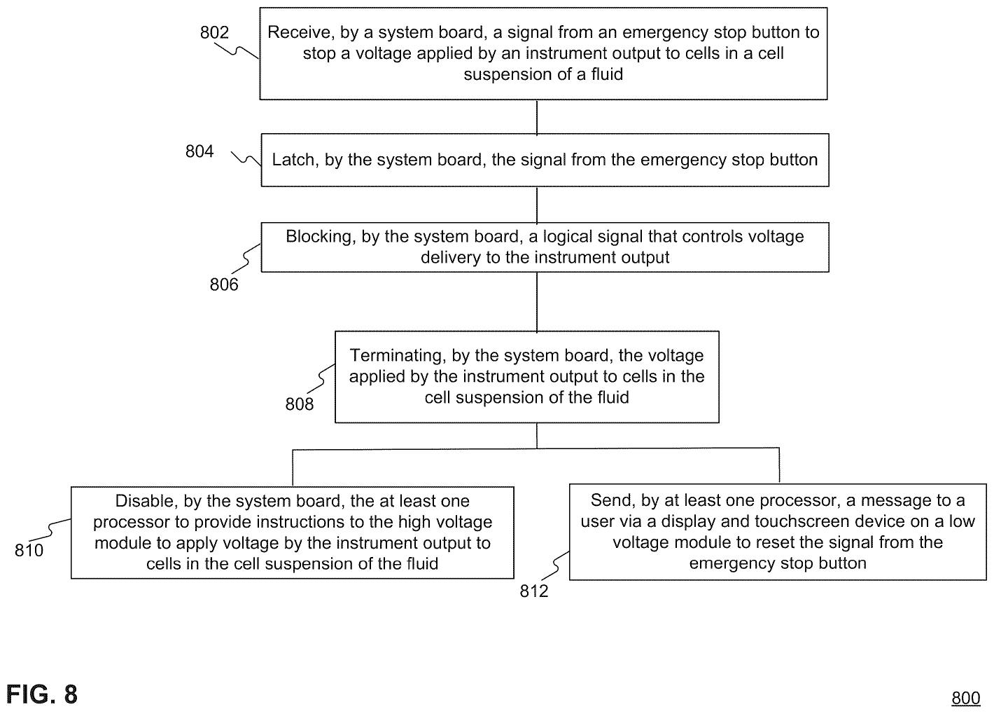

FIG. 8 illustrates exemplary block diagram for an exemplary method for electroporation of cells in a cell suspension in a fluid, consistent with some embodiments of the present disclosure. Method 800, as shown in FIG. 8, at block 802 may be performed by a system board that may receive a signal from an E-stop button 168 to stop the voltage applied by an instrument output 122 to cells in a cell suspension in a fluid. This signal may be a momentary signal generated at the instant a button of other signaling device is activated, and act as an interrupt for the electroporation process on cells of the cell of suspension of the fluid.

At block 804, method 800 may be performed by the system board that may latch the signal from the E-stop button 168. The signal may be latched once the momentary signal is input, without waiting for a clock signal. The signal may remain latched even if the input momentary signal is no longer being received.

At block 806, method 800 may be performed by the system board that may block a logical signal that may control voltage delivery to the instrument output. The at least one processor may send the logical signal to control voltage delivery to the instrument output.

At block 808, method 800 may be performed by the system board that may terminate the voltage applied by the instrument output 122 to the cells in the cell suspension in the fluid. This termination may happen near instantaneously, without waiting for further input from the processor 103.

At block 810, method 800 may be performed by the system board that may disable the processor 103 to provide instructions to the high voltage module 106 to apply the voltage by the instrument output 122 to cells in the cell suspension in the fluid.

At block 812, method 800 may be performed by the processor 103 that may send a message to a user via a display and touchscreen device 146 on a low voltage module 108 to reset the signal from the E-stop 168. This signal may be sent after block 808 is executed, but independent of block 810. Block 808 and block 810 may occur in parallel.

While the present disclosure has been shown and described with reference to particular embodiments thereof, it will be understood that the present disclosure can be practiced, without modification, in other environments. The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, or other optical drive media.

Computer programs based on the written description and disclosed methods are within the skill of an experienced developer. Various programs or program modules can be created using any of the techniques known to one skilled in the art or can be designed in connection with existing software. For example, program sections or program modules can be designed in or by means of. Net Framework,. Net Compact Framework (and related languages, such as Visual Basic, C, etc.), Java, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. An apparatus for electroporation of cells in a cell suspension in a fluid, the apparatus comprising:
a memory storing instructions;
a high voltage module including an instrument output configured to apply a voltage to cells in a cell suspension in a fluid;
a low voltage module including a user interface device;
a system board connected to the high voltage module, the low voltage module, and a single board computer;
the single board computer including at least one processor configured to (i) send instructions to control the high voltage module and the low voltage module, and (ii) receive instructions from the user interface device to regulate the voltage applied by the instrument output to the cells in the cell suspension in the fluid; and
an emergency stop button connected to the system board;
wherein the system board further includes an electronic circuit configured to execute the instructions to perform steps comprising:
receiving a signal from the emergency stop button to stop the voltage applied by the instrument output to the cells in the cell suspension in the fluid;
latching the signal from the emergency stop button;
blocking a logical signal that controls voltage delivery to the instrument output;
terminating the voltage applied by the instrument output to the cells in the cell suspension in the fluid; and
disabling the at least one processor from providing commands to the high voltage module.

2. The apparatus of claim 1, wherein the high voltage module includes a high voltage board and a pulse modulator board configured to execute a positive pulsing wave in the instrument output.

3. The apparatus of claim 2, wherein the high voltage board and the pulse modulator board are configured to generate the positive pulsing wave using at least one bank of capacitors.

4. The apparatus of claim 1, wherein the high voltage module includes a high voltage board and a pulse modulator board configured to execute a negative pulsing wave in the instrument output.

5. The apparatus of claim 4, wherein the high voltage board and the pulse modulator board generate the negative pulsing wave using at least one bank of capacitors.

6. The apparatus of claim 1, wherein a single board computer is configured to process electroporation results of the cells in the cell suspension in the fluid.

7. The apparatus of claim 1, wherein the single board computer is configured to manage the user interface.

8. The apparatus of claim 1, wherein the high voltage module includes a calibration port configured to simulate a positive and negative pulsing wave in the instrument output.

9. The apparatus of claim 1, wherein the electronic circuit includes a D flip-flop connecting to the emergency stop button via a preset pin and terminating the voltage applied by the instrument output to the cells in the cell suspension in the fluid via an inverted output pin.

10. The apparatus of claim 1, wherein the electronic circuit includes a D flip-flop connecting to the processor via an output pin, and the at least one processor connecting to the user interface device via a clock input pin to reset the signal from the emergency stop button.

11. A method of performing a safety step terminating the electroporation of cells in a cell suspension in a fluid, the method comprising:
receiving a signal from an emergency stop button to stop a voltage applied by an instrument output to cells in a cell suspension in a fluid;
latching the signal from the emergency stop button;
blocking a logical signal that controls voltage delivery to the instrument output;
terminating the voltage applied by the instrument output to cells in the cell suspension in the fluid; and
disabling the at least one processor from providing commands to the high voltage module.

12. The method of claim 11, wherein the high voltage module includes a high voltage board and a pulse modulator board to execute a positive pulsing wave in the instrument output.

13. The method of claim 12, wherein the high voltage board and the pulse modulator board generate the positive pulsing wave using at least one bank of capacitors.

14. The method of claim 11, wherein the high voltage module includes a high voltage board and a pulse modulator board to execute a negative pulsing wave in the instrument output.

15. The method of claim 14, wherein the high voltage board and the pulse modulator board generate the negative pulsing wave using at least one bank of capacitors.

16. The method of claim 11, wherein the single board computer is configured to manage the user interface.

17. The method of claim 11, wherein the high voltage module includes a calibration port configured to simulate a positive and negative pulsing wave in the instrument output.

18. The method of claim 11, wherein the electronic circuit includes a D flip-flop connecting to the at least one processor via an output pin, and the at least one processor connecting to the user interface device via a clock input pin to reset the signal from the emergency stop button.

19. The method of claim 11, wherein the electronic circuit includes a D flip-flop using an output pin connected to the single board computer managing the user interface, a clock input pin connected to the single board computer managing the user interface, and an input pin to reset the signal from the emergency stop button.

20. An apparatus for electroporation of cells in a cell suspension in a fluid, the apparatus comprising:
a memory for storing instructions;
a high voltage module including an instrument output configured to apply a voltage to cells in a cell suspension in a fluid;
a low voltage module including a user interface device;
a system board connected to the high voltage module, the low voltage module, and a single board computer;
the single board computer including at least one processor configured to (i) send instructions to control the high voltage module and the low voltage module, and (ii) receive instructions from the user interface device to regulate the voltage applied by the instrument output to the cells in the cell suspension in the fluid;
an emergency stop button connected to the system board;
a D flip-flop in the system board having a preset input pin connected to the emergency stop button, an output pin and a clock input pin connected to the low voltage module, and an inverted output pin connected to the high voltage module; and
wherein the D flip-flop is configured to execute the instructions to perform steps comprising:
receiving a signal from the emergency stop button to stop the voltage applied by the instrument output to cells in the cells suspension of the fluid;
latching the signal from the emergency stop button;
blocking a logical signal that controls voltage delivery to the instrument output;
terminating the voltage applied by the instrument output to cells in the cell suspension in the fluid; and
disabling the at least one processor from providing commands to the high voltage module.
